(19) United States
(12) Patent Application Publication
(10) Pub. No.: US 2012/0136302 A1
(43) Pub. Date: May 31, 2012

(54) Intelligent Close Loop Recognition Restoring Feedback Device

(75) Inventors: Chunxia Xu, Suzhou (CN); Kaiyou Ge, Suzhou (CN); Qunhua Li, Suzhou (CN); Ming Ji, Suzhou (CN)

(73) Assignee: MAQUET (SUZHOU) CO., LTD., Suzhou (CN)

(21) Appl. No.: 13/383,709

(22) PCT Filed: Feb. 9, 2011
(86) PCT No.: PCT/CN2011/000201

§ 371 Date: Jan. 12, 2012

(30) Foreign Application Priority Data
Oct. 18, 2010 (CN) 201010509742.9

(51) Int. Cl. A61M 5/14

(52) U.S. Cl. 604/131

(57) ABSTRACT

The invention provides an intelligent close loop recognition restoring feedback device including a back plate, a casing is disposed at a side of the back plate, and a covering plate is disposed on the casing. A rotating pivot is protruded from the middle of the covering plate and the middle of the casing. Two transverse rotating handles are disposed at an end of the rotating pivot, and the rotating handles are fixed by a plug cover. Two moving-sliding blocks are disposed in the casing, and the two moving-sliding blocks are connected to each other via tensile elastic elements. A cam is disposed between the two moving-sliding blocks, and each moving-sliding block is provided with a cylindrical pin. The two cylindrical pins are located at the same position on the moving-sliding blocks and abut against the edge of the cam. In the invention, the structure of the device is concise, and the device may easily detect the real-time operation conditions and reflect the message about whether the operation is right to the operator, and may prevent the operator from continuing operating the infusion transferring system under improper conditions. As a result, the safety of the device is guaranteed.

### FIELD OF THE INVENTION

The invention relates to an infusion transfer system art and, more particularly, to an intelligent close loop recognition restoring feedback device which can achieve the safety control.

### DESCRIPTION OF THE RELATED ART

A conventional way for transferring an infusion system is to hang an infusion bottle to a bracket that needs to be transferred. When a patient needs to leave a hospital bed for an examination room, the infusion system needs to be transferred along with the patient. As a result, the conventional transferring way is time consuming and laborious, and it is also easy to make mistake, increase the pain of the patient, delay the recovering process and even cause irretrievable results. Therefore, a fast and accurate way to transfer the infusion system is important to the safety and careful operation of patient, and a fast and safe way for transferring the infusion system is needed.

### BRIEF SUMMARY OF THE INVENTION

An objective of the invention is to provide an intelligent close loop recognition restoring feedback device which is transferred conveniently, is capable of reducing the pain of the patient, and allowing the patient to recover fast.

To solve the problem above, the invention provides an intelligent close loop recognition restoring feedback device including a back plate, a casing is disposed at a side of the back plate, and a covering plate is disposed on the casing. A rotating pivot is protruded from the middle of the covering plate and the middle of the casing. Two transverse rotating handles are disposed at an end of the rotating pivot, and the rotating handles are fixed by a plug cover. Two moving-sliding blocks are disposed in the casing, and the two moving-sliding blocks are connected to each other via tensile elastic elements. A cam is disposed between the two moving-sliding blocks, and each moving-sliding block is provided with a cylindrical pin. The two cylindrical pins are located at the same position on the moving-sliding blocks and abut against the edge of the cam.

To prevent the vertical bars from dropping during operating the device, the area of the cam with the same radius is larger than a half of the whole cam.

To make the device unable to be transferred and warn the user that the operation is improper when the device does not meet the requirement of transferring, a sliding block is disposed under each moving-sliding block, and a compression elastic element is disposed inside each sliding block to cooperate with the moving-sliding block. A cambered recess is disposed at the inner side of the head of each sliding block along the edge of the cam. Protrusions are disposed at the edge of the cam at the position corresponding to the cambered recesses.

To ensure that the device succeeds in finishing transferring the infusion system, as the cam starts to rotate, when a cylindrical pin on a moving-sliding block at one side is about to access the area of the cam with different radiuses, a protrusion is disposed at a shortest area that the sliding block on the moving-sliding block at the other side swept on the edge of the cam.

The protrusions are preferably located where the two protrusions and the center of the cam are in a line. More preferably, the two protrusions are symmetrical about the symmetric line of the cam.

To make the device have smaller level difference during transferring, a rotating reflection block is disposed above each sliding block in the middle of each moving-sliding block, and each rotating reflection block is connected to the corresponding moving-sliding block via a rotating pivot. The sliding block is provided with a step to cooperate with the rotating reflection block.

To reduce the abrasion of the cylindrical pin and facilitate the rotation of the cam, bearings are assembled at the surface of the cylindrical pins.

To make the operator can feel the operation is located in place during operating the device, two locating recesses are disposed at the intersecting positions of the edge of the cam and the symmetric line of the cam.

To make the device hold the vertical bars tightly, rubber mats are disposed on the moving-sliding block and the rotating reflection block at the ends away from the cam.

The beneficial effects of the invention lie in: the structure of the device in the invention is concise, and the device may easily detect the real-time operation conditions and reflect the message about whether the operation is right to the operator, and may prevent the operator from continuing operating the infusion transferring system under improper conditions. As a result, the safety of the device is guaranteed.

### BRIEF DESCRIPTION OF THE DRAWINGS

FIG. 1 is a schematic diagram showing the structure in an embodiment of the invention;

FIG. 2 is a schematic diagram showing inner structure in an embodiment of the invention;

FIG. 3 is a sectional diagram along B-B line in FIG. 1;

FIG. 4 is a schematic diagram showing the cam in an embodiment of the invention;

FIG. 5 is a schematic diagram showing the structure of the sliding block in an embodiment of the invention.

FIG. 6 and FIG. 7 are schematic diagrams showing the swept area of the sliding block during the rotating process in an embodiment of the invention.

FIG. 8 is a schematic diagram showing the first step of operating the device normally;

FIG. 9 is a schematic diagram showing the second step of operating the device normally;

FIG. 10 is a schematic diagram showing the third step of operating the device normally.

FIG. 11 is a schematic diagram showing the invention in an abnormal state (namely the device holds two vertical bars with different diameters); and

FIG. 12 is an enlarged diagram showing the partial area C in FIG. 10.

### SYMBOLS IN THE FIGS

1, back plate 2, cam 3, tensile elastic element 4, moving-sliding block 5, rotating reflection block 6, sliding block 7, protrusion 8, rubber mat 9, compression elastic element 10, cylindrical pin 11, bearing 12, handle 13, covering plate 14, casing 15, plug cover 16, symmetric line 17, rotating pivot 18, cambered recess 19, step 20, locating recess

### DETAILED DESCRIPTION OF THE EMBODIMENTS

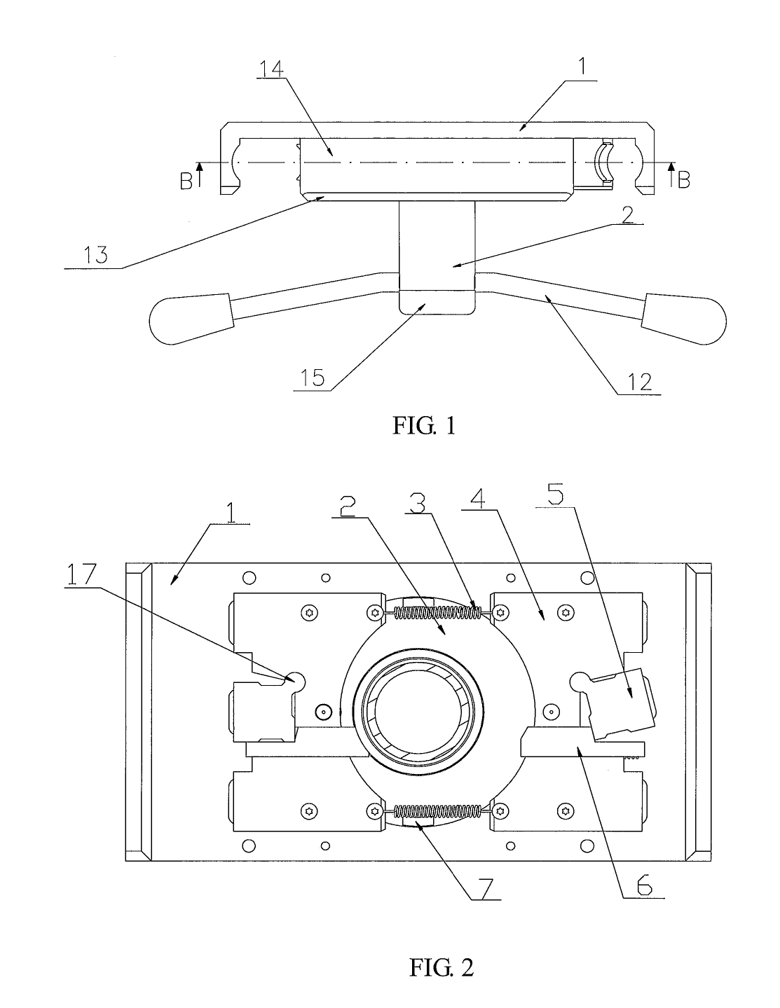

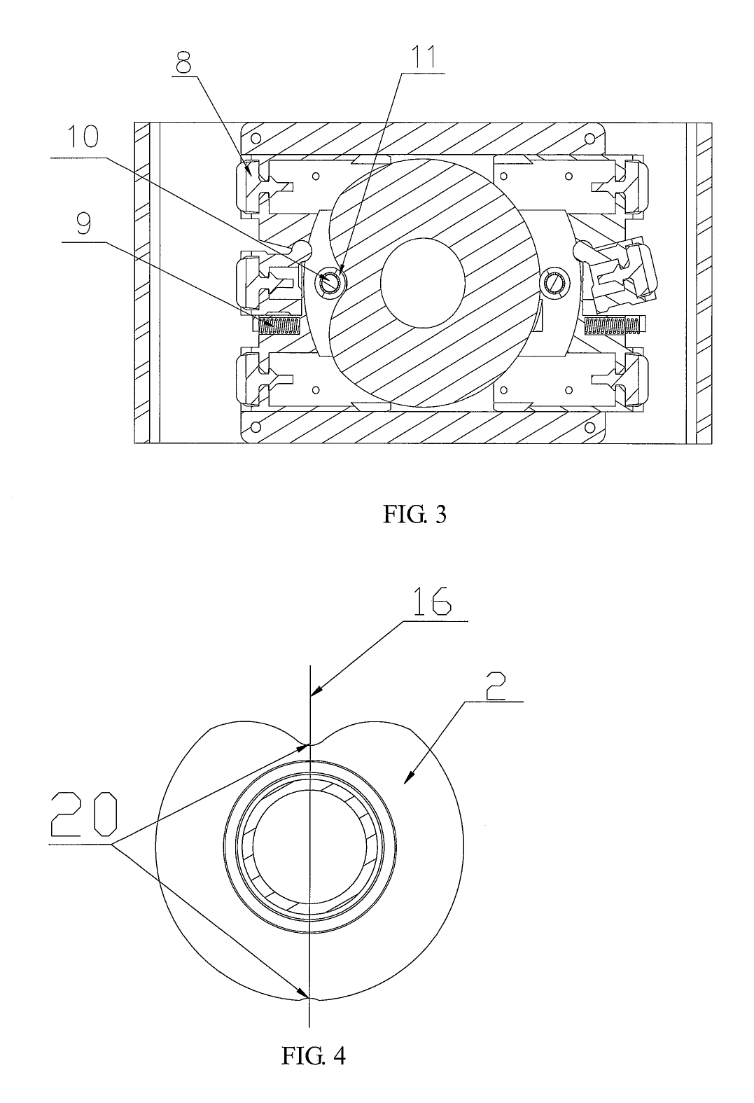

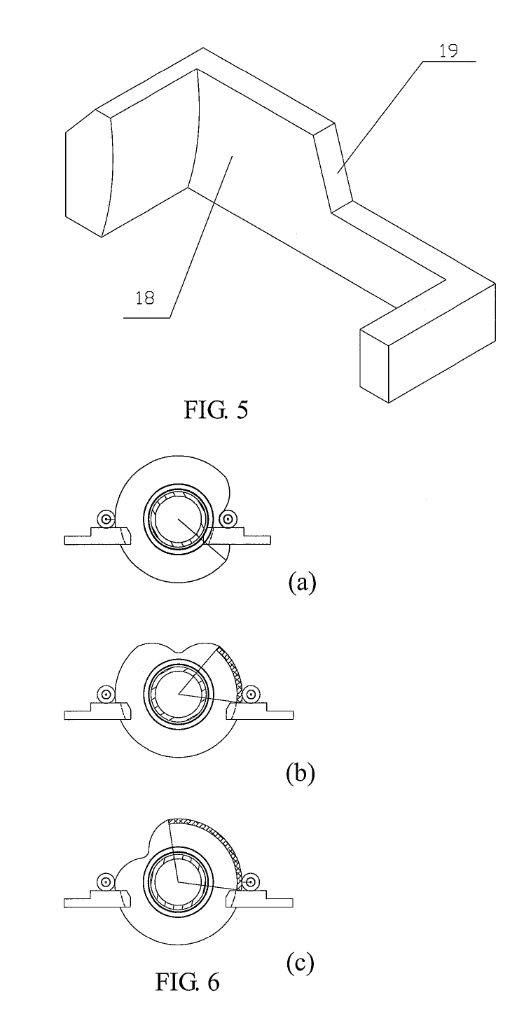

As shown in FIG. 1, FIG. 2, FIG. 3, FIG. 4 and FIG. 5, an embodiment of the invention provides an intelligent close loop recognition restoring feedback device including a back plate 1, a casing 14 is disposed at a side of the back plate 1, and a covering plate 13 is disposed on the casing 14. A rotating pivot 2 is protruded from the middle of the casing 14 and the middle of the covering plate 13. Two transverse rotating handles 12 are disposed at an end of the rotating pivot 2, and the rotating handles 12 are fixed by a plug cover 15. Two moving-sliding blocks 4 are disposed in the casing 14, and the two moving-sliding blocks 4 are connected to each other via tensile elastic elements 3. A cam 2 is disposed between the two moving-sliding blocks 4, and each moving-sliding block 4 is provided with a cylindrical pin 10. The two cylindrical pins 10 are located at the same position on the moving-sliding blocks 4 and abut against the edge of the cam 2, and the area of the cam 2 with the same radius is larger than a half of the whole cam 2. Two sliding blocks 6 are disposed under the moving-sliding blocks 4, and a compression elastic element 9 is disposed inside each of the sliding blocks 6, respectively, to cooperate with the moving-sliding blocks 4. A cambered recess 18 is disposed at the inner side of the head of each sliding block 6 along the edge of the cam 2. Protrusions 7 are disposed at the edge of the cam 2 at the position corresponding to the cambered recesses 18. A rotating reflection block 5 is disposed above each sliding block 6 in the middle of each moving-sliding block 4, and each rotating reflection block 5 is connected to the corresponding moving-sliding block 4 via a rotating pivot 17. The sliding block 6 is provided with a step 19 to cooperate with the rotating reflection block 5. Bearings 11 are disposed at the surface of the cylindrical pins 10, and two locating recesses 19 are disposed at the edge of the cam 2 along the symmetric line 16 of the cam 2. Rubber mats 8 are disposed on the moving-sliding block 4 and the rotating reflection block 5 on the ends away from the cam 2.

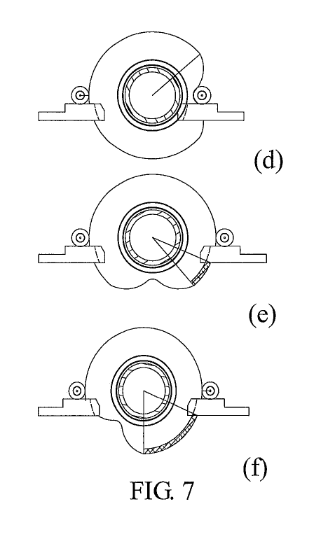

As shown in FIG. 6 and FIG. 7, FIG. 6(a) and FIG. 7(d) show a start-rotating position, FIG. 6(b) and FIG. 7(e) show a middle position, and FIG. 6(c) and FIG. 7(f) show a finish-rotating position. As the cam 2 starts to rotate, when a cylindrical pin 10 on a moving-sliding block 4 at one side is about to access the area of the cam 2 with different radiuses, a protrusion 7 is disposed at a shortest area that the sliding block 6 on the moving-sliding block 4 at the other side swept on the edge of the cam 2. Another protrusion 7 is disposed at a symmetrical area of the shortest area about the symmetric line 16. The protrusions 7 are preferably located where the two protrusions and the center of the cam are in a line. To operate conveniently, the two protrusions 7 are symmetrical about the symmetric line 16 of the cam 7. The swept area in FIG. 6 is larger than the swept area in FIG. 7, and to prevent the pole from dropping from the device no matter how the device is rotated, the overlapping areas in FIG. 6 and FIG. 7 which are the areas in FIG. 7 are selected. The symmetrical areas of the areas in FIG. 7 about the symmetric line 16 are provided with protrusions 7, too.

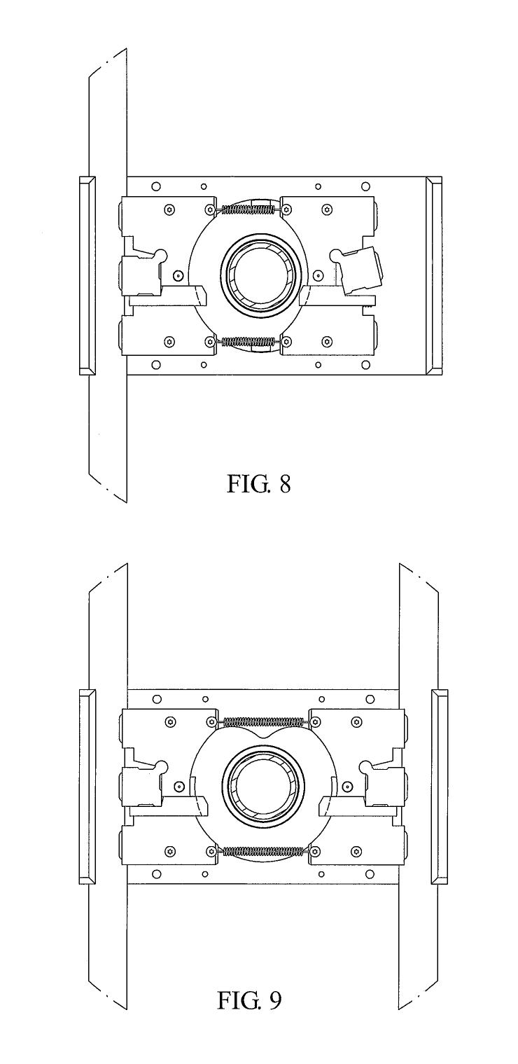

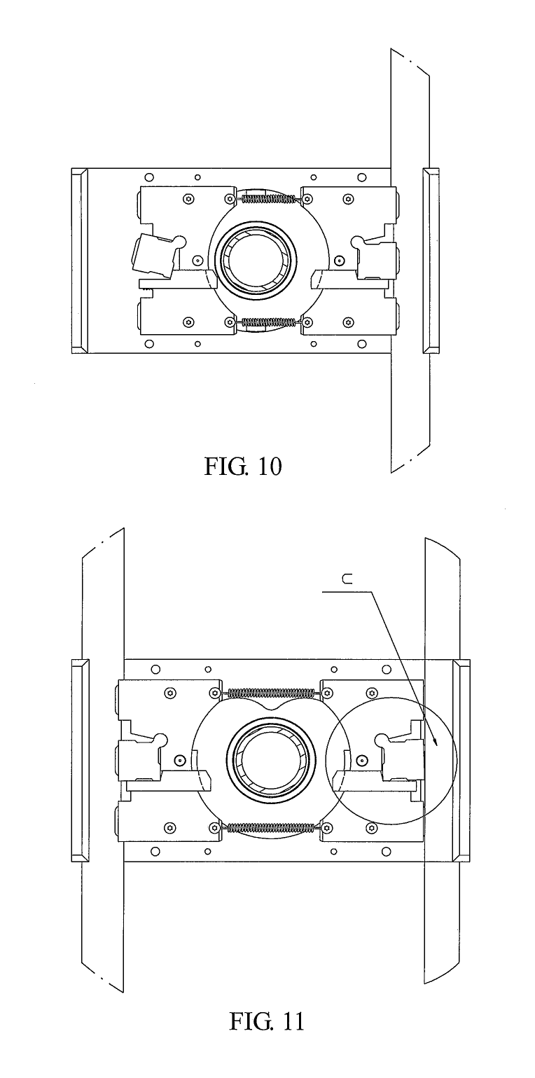

FIG. 8, FIG. 9 and FIG. 10 show basic steps for operating the device normally. When the infusion system needs to be transferred, a vertical bar which has the same diameter with a vertical bar at the left side is disposed at the right side, the rotating handle 12 is rotated to drive the cam 2, and when the cam 2 rotates to make the cylindrical pin 10 and the bearings 11 access the area of the cam with the same radius, the rotating reflection block 5 at the right side is pressed by an external force to drive the sliding block 6 at the right side to be pressed, and the rotating reflection block 5 at the right side moves towards the rotating cam 2 along a guiding slot of the moving-sliding block 4 under the force of the compression elastic element 9. Due to the cooperation of the cylindrical pins 10 and the bearings 11 disposed on the moving-sliding block 4 and the tensile elastic element 3, the cam 2 drives the moving-sliding blocks 4 at both sides to move transversely and oppositely. The cam 2 is provided with two protrusions 7 with the interval of 180 degrees, and when the cam 2 rotates for about 90 degrees, and the rotating reflection blocks 5 at both sides are pressed completely by an external force, the two protrusions 7 with the interval of 180 degrees on the edge of the cam 2 succeed in passing the cambered recesses 18 at the two sliding blocks 6. At that moment, the operator can operate the infusion transferring system correctly and smoothly. When the rotating handle 12 rotates for 180 degrees, the transferring process is finished safely.

FIG. 11 shows the situation that the safety transfer requirement is not met. Namely, in FIG. 11 two vertical bars disposed at two sides of the device have different diameters. The rotating reflection block 5 is not pressed by the external force completely, namely the press force is not enough, the two protrusions 7 with the interval of 180 degrees at the edge of the cam 2 cannot pass the cambered recesses 18 on the sliding blocks 6. The rotating handle 12 cannot rotate towards the required rotating direction, and this prevents the operator from continuing operating, until the operator operates the device under the proper condition.

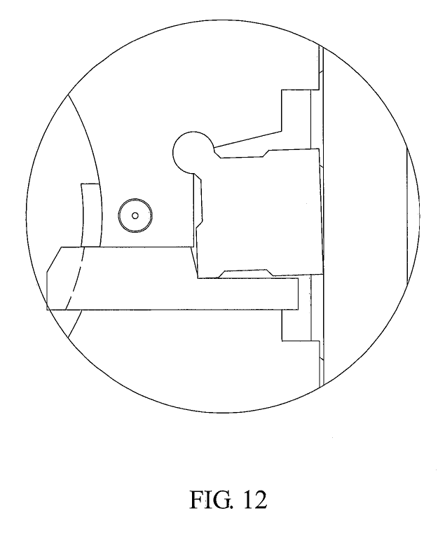

As shown in FIG. 12, when the pressure of the rotating reflection block 5 is not enough (namely the diameter of the vertical bars cannot meet the requirement), the sliding block 6 cannot be pressed completely, and the protrusions 7 cannot pass the cambered recess 18 smoothly, which forbids the operation from being performed normally.

Although the invention has been described as above in reference to some typical embodiments, it is to be understood that the terms used therein are just illustrative and exemplary rather than restrictive. Since the invention can be applied in various forms without departing from the spirit or principle of the invention, it is to be understood that the abovementioned embodiments will not be limited to any specific details mentioned above, rather, they should be construed broadly in the spirit or concept of the invention defined by the appended claims. Therefore, the present invention aims to cover all the modifications or variations falling within the protection scope defined by the appended claims.

What is claimed is:

1. An intelligent close loop recognition restoring feedback device, comprising a back plate (1), a casing (14) is disposed at a side of the back plate (1), and a covering plate (13) is disposed on the casing (14), a rotating pivot (2) is protruded from the middle of the casing (14) and the middle of the covering plate (13), and two transverse rotating handles (12) are disposed at an end of the rotating pivot (2), and the rotating handles are fixed by a plug cover (15), two moving-sliding blocks (4) are disposed in the casing (14), and the two moving-sliding blocks (4) are connected to each other via tensile elastic elements (3), a cam (2) is disposed between the two moving-sliding blocks (4), and each moving-sliding block (4) is provided with a cylindrical pin (10), the two cylindrical pins (10) are located at the same position on the moving-sliding blocks (4) and abut against the edge of the cam (2).

2. The intelligent close loop recognition restoring feedback device according to claim 1, wherein the area of the cam (2) with the same radius is larger than a half of the whole cam (2).

3. The intelligent close loop recognition restoring feedback device according to claim 2, wherein a sliding block (6) is disposed under each moving-sliding block (4), and a compression elastic element (9) is disposed inside each sliding block (4) to cooperate with the moving-sliding block (4), a cambered recess (18) is disposed at the inner side of the head of each sliding block (6) along the edge of the cam (2), respectively, and protrusions (7) are disposed at the edge of the cam (2) at the position corresponding to the cambered recesses (18).

4. The intelligent close loop recognition restoring feedback device according to claim 3, wherein as the cam (2) starts to rotate, when a cylindrical pin (10) on a moving-sliding block (4) at one side is about to access the area of the cam (2) with different radiuses, a protrusion (7) is disposed at a shortest area that the sliding block (6) on the moving-sliding block (4) at the other side swept on the edge of the cam (2).

5. The intelligent close loop recognition restoring feedback device according to claim 3 or 4, wherein the protrusions (7) are located where the two protrusions (7) and the center of the cam (2) are in a line.

6. The intelligent close loop recognition restoring feedback device according to claim 5, wherein the two protrusions (7) are symmetrical about the symmetric line (16) of the cam (2).

7. The intelligent close loop recognition restoring feedback device according to claim 3 or 4, wherein a rotating reflection block (5) is disposed above each sliding block (6) in the middle of each moving-sliding block (4), and each rotating reflection block (5) is connected to the corresponding moving-sliding block (4) via a rotating pivot (17), the sliding block (6) is provided with a step (19) to cooperate with the rotating reflection block (5).

8. The intelligent close loop recognition restoring feedback device according to claim 7, wherein bearings (11) are assembled at the surface of the cylindrical pins (10).

9. The intelligent close loop recognition restoring feedback device according to claim 7, wherein two locating recesses (19) are disposed at the intersecting positions of the edge of the cam (2) and the symmetric line (16) of the cam (2).

10. The intelligent close loop recognition restoring feedback device according to claim 1, wherein rubber mats (8) are disposed on the moving-sliding block (4) and the rotating reflection block (5) at the ends away from the cam (2).
